(19) United States
(12) Patent Application Publication
(10) Pub. No.: US 2011/0263940 A1
(43) Pub. Date: Oct. 27, 2011

(54) ENDOSCOPE APPARATUS

(75) Inventor: Hiroshi YAMAGUCHI, Kanagawa (JP)

(73) Assignee: FUJIFILM CORPORATION, Tokyo (JP)

(21) Appl. No.: 13/093,638

(22) Filed: Apr. 25, 2011

(30) Foreign Application Priority Data
Apr. 26, 2010 (JP) 2010-100854

(51) Int. Cl. A61B 1/06

(52) U.S. Cl. 600/163

(57) ABSTRACT

An endoscope apparatus comprising an irradiating section that irradiates the target with the irradiation light containing light in a first wavelength region, which reaches a first penetration depth within the target, and light in a second wavelength region, which reaches a second penetration depth within the target; an optical system that focuses, at substantially the same position in a direction of an optical axis thereof, first returned light from a position at a distance of the first penetration depth from a surface of the target in a direction of emission of the light in the first wavelength region and second returned light from a position at a distance of the second penetration depth from the surface of the target in a direction of emission of the light in the second wavelength region; and a light receiving section that receives the first returned light and the second returned light.

### BACKGROUND

1. Technical Field

The present invention relates to an endoscope apparatus. The contents of the following Japanese patent application are incorporated herein by reference,
NO. 2010-100854 filed on Apr. 26, 2010.

2. Related Art

An observation apparatus is known that optically obtains information at different depths in an organism, as shown in Patent Documents 1 and 2, for example.
Patent Document 1: Japanese Patent Application Publication No. 2005-99430
Patent Document 2: Japanese Patent Application Publication No. 2007-47228

A plurality of points can be measured in the depth direction by changing the depth of the focal point of irradiation light for each wavelength using an objective lens, but the measurement can be performed for only a single point in a plane perpendicular to the optical axis of the objective lens. Therefore, there is a problem that image information within a plane perpendicular to the image capturing direction cannot be captured in a single attempt while maintaining high resolution in the depth direction.

### SUMMARY

In order to solve the above problems, according to a first aspect related to the innovations herein, provided is an endoscope apparatus that captures an image of a target using returned light from the target irradiated with irradiation light, the endoscope apparatus comprising an irradiating section that irradiates the target with the irradiation light containing light in a first wavelength region, which reaches a first penetration depth within the target, and light in a second wavelength region, which reaches a second penetration depth within the target; an optical system that focuses, at substantially the same position in a direction of an optical axis thereof, first returned light from a position at a distance of the first penetration depth from a surface of the target in a direction of emission of the light in the first wavelength region and second returned light from a position at a distance of the second penetration depth from the surface of the target in a direction of emission of the light in the second wavelength region; and a light receiving section that receives the first returned light and the second returned light focused by the optical system.

The endoscope apparatus may further comprise an image generating section that generates images within the target at different positions in the direction of the optical axis of the optical system, based on the first returned light and the second returned light received by the light receiving section.

The irradiating section may include a light source that emits (i) illumination light for illuminating the surface of the target, (ii) the light in the first wavelength region, which is light in a narrower wavelength region than the illumination light, and (iii) the light in the second wavelength region, which is light in a narrower wavelength region than the illumination light, the light receiving section may further receive returned light from the target irradiated with the illumination light, and the image generating section may further generate an image of the surface of the target based on the returned light from the target irradiated with the illumination light.

The illumination light may be light in a visible region, the light in the first wavelength region may be light in a blue wavelength region, and the first returned light may be light in substantially the same wavelength region as the first wavelength region.

The light in the second wavelength region may be light in a longer wavelength region than the first wavelength region, and the second returned light may be light in substantially the same wavelength region as the second wavelength region.

The light in the second wavelength region may be light in an infrared region.

The endoscope apparatus may further comprise a first wavelength filter that passes light in a wavelength region of the first returned light and a second wavelength filter that passes light in a wavelength region of the second returned light, and the light receiving section may include a first light receiving element that receives light passed by the first wavelength filter and a second light receiving element that receives light passed by the second wavelength filter.

A plurality of the first wavelength filters and a plurality of the second wavelength filters may be provided and arranged two-dimensionally, and a plurality of the first light receiving elements and a plurality of the second light receiving elements may be provided at positions corresponding respectively to the first wavelength filters and the second wavelength filters.

The target may include a luminescent substance that emits luminescent light as a result of being excited by the light in the second wavelength region contained in the irradiation light, and the optical system may focus the first returned light and the second returned light, which is the luminescent light emitted by the luminescent substance, at substantially the same position in the direction of the optical axis.

The luminescent substance may emit luminescent light as a result of being excited by the light in the first wavelength region in the irradiation light and also emit luminescent light as a result of being excited by the light in the second wavelength region in the irradiation light, and the optical system may focus the first returned light and the second returned light, which are both in the wavelength region of the luminescent light emitted by the luminescent substance, at substantially the same position in the direction of the optical axis.

The light in the first wavelength region and the light in the second wavelength region included in the irradiation light respectively excite different luminescent substances in the target.

The endoscope apparatus may further comprise an injecting section that injects each of a plurality of the luminescent substances into the target.

The summary clause does not necessarily describe all necessary features of the embodiments of the present invention. The present invention may also be a sub-combination of the features described above.

### BRIEF DESCRIPTION OF THE DRAWINGS

FIG. 1 shows an exemplary endoscope apparatus 10 according to an embodiment of the present invention.

FIG. 2 is a schematic view of an exemplary configuration of the image capturing section 124, along with the analyte 20.

FIG. 3 is a schematic view of exemplary configurations of a light emitting system and an image capturing system in the insertion section 120.

FIG. 4 is a schematic view of exemplary configurations of the wavelength filter section 330 and the light receiving section 320.

FIG. 5 shows exemplary image capturing timings of the illumination light images and narrow-band light images by the image capturing section 124.

FIG. 6 shows an exemplary image on the screen of the display apparatus 140.

FIG. 7 shows another exemplary narrow-band light image generated by the image generating section 102.

### DESCRIPTION OF EXEMPLARY EMBODIMENTS

Hereinafter, some embodiments of the present invention will be described. The embodiments do not limit the invention according to the claims, and all the combinations of the features described in the embodiments are not necessarily essential to means provided by aspects of the invention.

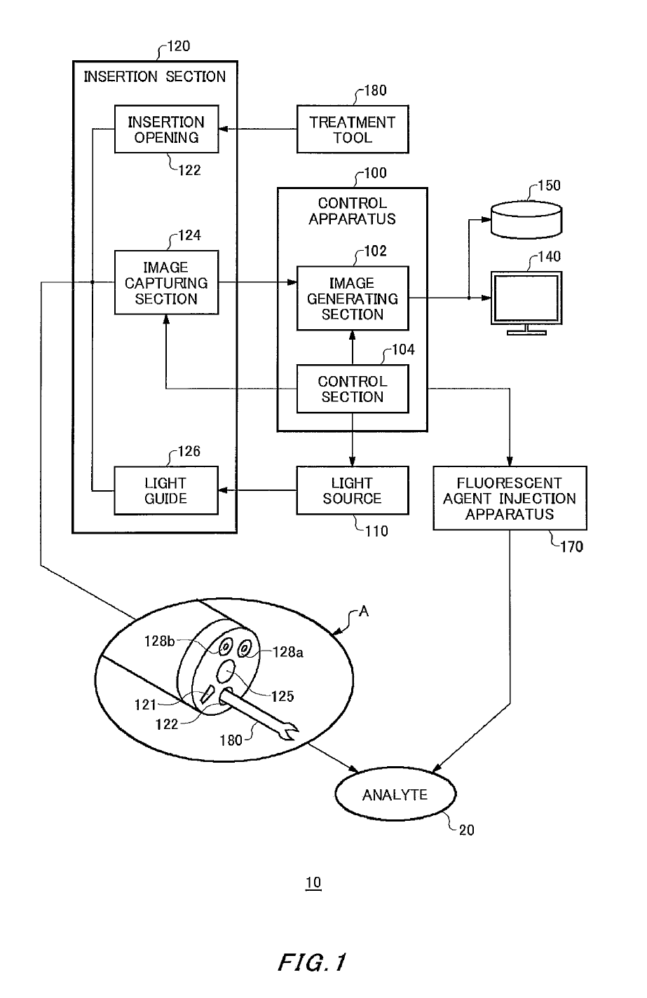

FIG. 1 shows an exemplary endoscope apparatus 10 according to an embodiment of the present invention. The endoscope apparatus 10 of the present embodiment captures an image of an analyte 20, which is a living creature, for example. Specifically, the endoscope apparatus 10 captures an image of the analyte 20 using returned light from the analyte 20 irradiated with irradiation light.

In the present embodiment, the endoscope apparatus 10 captures images within the analyte 20 at different depths. For example, the endoscope apparatus 10 may irradiate the analyte 20 with special observation light in different wavelength regions. Specifically, the endoscope apparatus 10 irradiates the analyte 20 with special observation light including light in the blue wavelength region and light in the red wavelength region as primary components. The light in the blue wavelength region incident to the analyte 20 is reflected and/or scattered by objects relatively near the surface layer of the analyte 20, and therefore light in the blue wavelength region is returned. On the other hand, the light in the red wavelength region incident to the analyte 20 can travel relatively deeply into the analyte 20, and is therefore reflected and/or scattered by objects deep within the analyte 20, causing light in the red wavelength region to be returned.

The image capturing optical system of the endoscope apparatus 10 has an axial chromatic aberration such that returned light in the blue wavelength region from the observed position in the analyte 20, resulting from the light in the blue wavelength region, and returned light in the red wavelength region from the observed position in the analyte 20, resulting from the light in the red wavelength region, are focused at substantially the same position on the optical axis of the image capturing optical system. The endoscope apparatus 10 can perform a one-shot capture of special observation light images at different depths by capturing a fluorescent light image of the analyte 20 via this image capturing optical system.

The analyte 20 in the present embodiment may be an internal organ such as an intestinal tube, including the stomach, large intestine, colon, or the like inside a living creature such as a person, for example. The analyte 20 may be the outside or the inside lining of an internal organ. In the present embodiment, the region serving as the image capturing target of the endoscope apparatus 10 is referred to as the analyte 20. The endoscope apparatus 10 includes an insertion section 120, a light source 110, a control apparatus 100, a fluorescent agent injection apparatus 170, a recording apparatus 150, a display apparatus 140, and a treatment tool 180. An enlarged view of the tip of the insertion section 120 is shown in section A of FIG. 1.

The insertion section 120 includes an insertion opening 122, an image capturing section 124, and a light guide 126. The tip of the insertion section 120 includes an objective lens 125 as a portion of the image capturing section 124. The objective lens 125 is included in the image capturing optical system. The tip of the insertion opening 122 includes a nozzle 121.

The insertion section 120 is inserted into an organism. A treatment tool 180, such as forceps, for treating the analyte 20 is inserted into the insertion opening 122. The insertion opening 122 guides the treatment tool 180 inserted thereto to the tip. The treatment tool 180, which is exemplified by forceps, can have a variety of tip shapes. The nozzle 121 discharges water or air toward the analyte 20.

The light guide 126 guides the light emitted by the light source 110 to the irradiating section 128. The light guide 126 can be realized using optical fiber, for example. The irradiating section 128 emits the light guided by the light guide 126 toward the analyte 20. The image capturing section 124 receives the light returning from the analyte 20 via the objective lens 125 to capture an image of the analyte 20.

The image capturing section 124 can capture illumination light images and special observation light images. The image capturing section 124 captures an illumination light image of the analyte 20 using illumination light with a relatively broad spectrum in the visible light band. When capturing an illumination light image, the light source 110 emits substantially white light in the visible light region. The illumination light includes light in the red wavelength region, the green wavelength region, and the blue wavelength region, for example. The illumination light emitted by the light source 110 is emitted toward the analyte 20 from the irradiating section 128a via the light guide 126. The objective lens 125 receives, as the returned light, light in the visible light region expanded to have substantially the same wavelength region as the illumination light, as a result of the analyte 20 reflecting and scattering the illumination light. The image capturing section 124 captures an image via the objective lens 125 using the returned light from the analyte 20. The light source 110 may include an illumination light source that generates the illumination light. The illumination light source may be a discharge lamp such as a xenon lamp, a semiconductor light emitting element such as an LED, or the like.

The special observation light images may be narrow-band light images captured when the analyte 20 is irradiated with narrow-band light. For example, the irradiating section 128a may irradiate the analyte 20 with narrow-band blue light in the blue wavelength region, as an example of the special observation light. The narrow-band blue light may be light in a narrower band than light in the blue wavelength region included in the illumination light. The majority of the emitted narrow-band blue light is reflected and scattered by the surface layer of the analyte 20, and becomes incident to the objective lens 125 as returned light. As a result, a narrow-band blue light image is obtained in which the surface layer of the analyte 20 is enhanced.

The irradiating section 128a irradiates the analyte 20 with narrow-band red light in the red wavelength region, as an example of the special observation light. The narrow-band red light may be light in a narrower band than the light in the red wavelength region included in the illumination light. The emitted narrow-band red light is not significantly reflected or scattered by the surface layer of the analyte 20, and therefore travels relatively deeply into the analyte 20. The light is reflected and scattered by an object deep in the analyte 20, and becomes incident to the objective lens 125 as the returned light. Accordingly, by irradiating the analyte 20 with the narrow-band red light, a narrow-band red light image is obtained that displays information concerning deep portions of the analyte 20. In addition to the narrow-band blue light and the narrow-band red light, narrow-band green light in the green wavelength region or narrow-band infrared light in the infrared wavelength region may be used as the special observation light. The narrow-band light can define light in a narrower wavelength region than the wavelength region of the illumination light irradiating the surface of the analyte 20. For example, the narrow-band light can be narrow-band light over a wavelength region spanning from infrared to red or narrow-band light over a wavelength region spanning from ultra violet to blue.

In the present embodiment, narrow-band blue light images and narrow-band red light images are captured as the special observation light images. The narrow-band blue returned light resulting from irradiation with the narrow-band blue light is light from relatively near a surface layer of the analyte 20, and the narrow-band red returned light resulting from irradiation with the narrow-band red light is light from a relatively deep portion of the analyte 20. Therefore, the narrow-band blue returned light and the narrow-band red returned light are returned from different positions within the analyte 20. The image capturing optical system of the endoscope apparatus 10 focuses each type of narrow-band light at substantially the same position on the optical axis of the image capturing optical system. The image capturing section 124 can capture narrow-band light images at different positions in the analyte 20 by receiving fluorescent light using light receiving elements provided at the focal position.

In addition to the narrow-band light images, the special observation light images may be luminescent images captured using luminescent light, which is an example of returned light from the analyte 20. Fluorescent and phosphorescent light are included in the scope of the luminescent light. The luminescent light can be generated by photoluminescence achieved using excitation light or the like. If the luminescent light is generated from the analyte 20 indirectly using a chemical process and/or a thermal process when the analyte 20 is irradiated, the image capturing section 124 can capture an image of the analyte 20 using this luminescent light. Accordingly, light generated via processes such as chemical luminescence or thermoluminescence is included in the scope of the luminescent light.

When capturing a fluorescent light image of the analyte 20, the light source 110 generates excitation light. The excitation light generated by the light source 110 is emitted toward the analyte 20 from the irradiating section 128b, via the light guide 126. A fluorescent substance in the analyte 20 is excited by the excitation light, and therefore emits fluorescent light. The image capturing section 124 captures the fluorescent light image of the analyte 20 using the fluorescent returned light. As shown in FIG. 1, the irradiating section 128a and the irradiating section 128b may be provided at different positions on the tip, but can instead be provided at the same position on the insertion section 120 to function as an irradiating section providing both illumination light and excitation light. The light source 110 may include an excitation light source that generates the excitation light. The excitation light source may be a semiconductor light emitting element such as an LED or a diode laser. As other examples, the excitation light source can use a laser with a variety of lasing media such as a diode laser, a fixed laser, or a liquid laser.

The fluorescent substance is an example of a luminescent substance. The fluorescent substance may be injected into the analyte 20 from the outside. The fluorescent substance may be indo cyanine green (ICG), for example. The fluorescent agent injection apparatus 170 may inject the ICG into the blood vessels of an organism using an intravenous injection. The amount of ICG that the fluorescent agent injection apparatus 170 injects into the analyte 20 is controlled by the control apparatus 100 to maintain a substantially constant concentration of ICG in the organism. The ICG is excited by infrared rays with a wavelength of 780 nm, for example, and generates fluorescent light whose primary spectrum is in a wavelength region of 830 nm. In the present embodiment, the image capturing section 124 captures fluorescent light images of the analyte 20 using the fluorescent light generated by the ICG. Infrared rays with a wavelength of 780 nm can penetrate deeper into the analyte 20 than light in the blue wavelength region or the green wavelength region, for example. The fluorescent light in a relatively long wavelength region generated from a deep portion is emitted from the analyte 20 as returned light, with relatively little scattering. Accordingly, the fluorescent light from the ICG can be used to obtain information from a deeper portion than returned light obtained by irradiation with narrow-band blue light or narrow-band green light.

The fluorescent substance can be a substance other than ICG. If structural components, such as cells, of the analyte 20 already contain a fluorescent substance, the image capturing section 124 may capture the fluorescent light image of the analyte 20 using the organism's own fluorescent light as the returned light. For example, the fluorescent substance contained in the structural components, such as cells, of the analyte 20 may be reduced NADH (nicotinamide adenine dinucleotide). NADH is excited by light with a wavelength of 340 nm in the ultra violet wavelength region to emit fluorescent light whose primary spectrum is in the 450 nm wavelength region. In addition to NADH, the fluorescent substance in an organism may be FAD (flavin adenine dinucleotide) or collagen contained in connective tissue or the like of the organism, for example.

Each type of fluorescent substance may be injected into the analyte 20 from the outside or may be already present in the analyte 20. The fluorescent substance may be a combination of a fluorescent substance injected into the analyte 20 from the outside and a fluorescent substance already present in the analyte 20. Three or more types of fluorescent substances may be used. The fluorescent agent injection apparatus 170 can inject the analyte 20 with each of two or more types of fluorescent substances.

The control apparatus 100 includes an image generating section 102 and a control section 104. The control section 104 controls the image capturing section 124 and the light source 110 and uses the image capturing section 124 to capture the illumination light images and the special observation light images. Specifically, the control section 104 causes the image capturing section 124 to switch over time between capturing the illumination light images and capturing the special observation light images.

The image generating section 102 generates an output image to be output to the outside, based on the illumination light images and the special observation light images captured by the image capturing section 124. For example, the image generating section 102 may output the generated output image to at least one of the recording apparatus 150 and the display apparatus 140. More specifically, the image generating section 102 generates an image from the plurality of images captured by the image capturing section 124, and outputs this image to at least one of the recording apparatus 150 and the display apparatus 140. The image generating section 102 may output the output image to at least one of the recording apparatus 150 and the display apparatus 140 via a communication network such as the Internet.

The display apparatus 140 displays images including the special observation light images and the illumination light images generated by the image generating section 102. The recording apparatus 150 records the images generated by the image generating section 102 in a non-volatile recording medium. For example, the recording apparatus 150 may store the images in a magnetic recording medium such as a hard disk or in an optical recording medium such as an optical disk.

The endoscope apparatus 10 described above can perform wavelength separation on the light from different depths in the analyte 20 to capture an image in a single shot. Therefore, the endoscope apparatus 10 can provide an observer with an image in which positional relationships in the depth direction are easily understood.

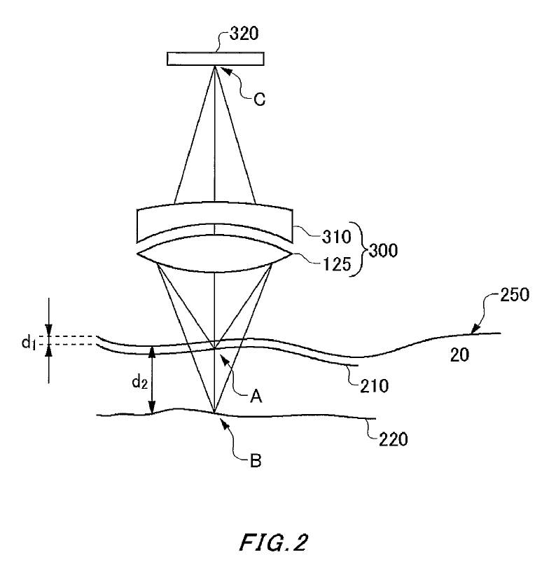

FIG. 2 is a schematic view of an exemplary configuration of the image capturing section 124, along with the analyte 20. The image capturing section 124 includes an image capturing optical system 300 and a light receiving section 320. The image capturing optical system 300 includes the objective lens 125 and a chromatic aberration correcting optical system 310. The following description focuses on the image capturing optical system 300 and the light receiving section 320 of the image capturing section 124.

In the present embodiment, narrow-band blue light and narrow-band red light are used as the special observation light. A blood vessel 210 such as a capillary blood vessel relatively near the surface layer can be the target of image capturing using the narrow-band blue light. A blood vessel 220 in a relatively deep portion can be the target of image capturing using the narrow-band red light.

In this case, the irradiating section 128a irradiates the analyte 20 with irradiation light including narrow-band blue light that reaches to a first penetration depth in the analyte 20 and narrow-band red light that reaches to a second penetration depth in the analyte 20. The narrow-band blue light and the narrow-band red light are respectively examples of light in a first wavelength region and light in a second wavelength region. The first penetration depth and second penetration depth are determined by the light scattering characteristics of the analyte 20 for each wavelength, and the first and second wavelength regions. In the present embodiment, the second penetration depth d2, which is the penetration depth of the narrow-band red light into the analyte 20, is greater than the first penetration depth d1, which is the penetration depth of the narrow-band blue light into the analyte 20.

The narrow-band blue light reaches point A at the first penetration depth d1 from the surface 250, and is reflected by an object at point A back toward the image capturing optical system 300 as narrow-band blue returned light. The narrow-band red light reaches point B at the second penetration depth d2 from the surface 250, and is reflected by an object at point B back toward the image capturing optical system 300 as narrow-band red returned light.

The image capturing optical system 300 has optical characteristics to substantially focus both the narrow-band blue light emitted from point A and the narrow-band red light emitted from point B at point C. The chromatic aberration correcting optical system 310 corrects the chromatic aberrations of the narrow-band blue light emitted from point A and the narrow-band red light emitted from point B. Here, Z represents the difference between the position on the optical axis of the image capturing optical system 300 at which the narrow-band blue light from point A is focused by the image capturing optical system 300 and the position on the optical axis of the image capturing optical system 300 at which the narrow-band red light from point B is focused by the image capturing optical system 300. The chromatic aberration correcting optical system 310 may be any optical system that can decrease the Z value of the image capturing optical system 300 more than if the chromatic aberration correcting optical system 310 were not used.

In this way, the image capturing optical system 300 can focus, at substantially the same position in the direction of the optical axis, both (i) the first returned light from the position that is the first penetration depth d1 from the surface 250 of the analyte 20 along the emission direction of the light in the first wavelength region and (ii) the second returned light from the position that is the second penetration depth d2 from the surface 250 of the analyte 20 along the emission direction of the light in the second wavelength region. The light in the first wavelength region may be light in the blue wavelength region. In this case, the returned light includes light in a blue wavelength region that is substantially equal to the first wavelength region. The light in the second wavelength region may be light in a longer wavelength region than the first wavelength region. For example, if the light in the second wavelength region is light in the red wavelength region, the returned light contains light in a red wavelength region that is substantially equal to the second wavelength region. In the present embodiment, the first returned light and the second returned light respectively correspond to narrow-band blue returned light and narrow-band red returned light.

The light receiving section 320 is provided near point C in the direction of the optical axis of the image capturing optical system 300. As a result, the light receiving elements of the light receiving section 320 near point C can receive the narrow-band blue returned light and the narrow-band red returned light focused by the image capturing optical system 300. In this way, the light receiving section 320 can receive the first returned light and the second returned light focused by the image capturing optical system 300.

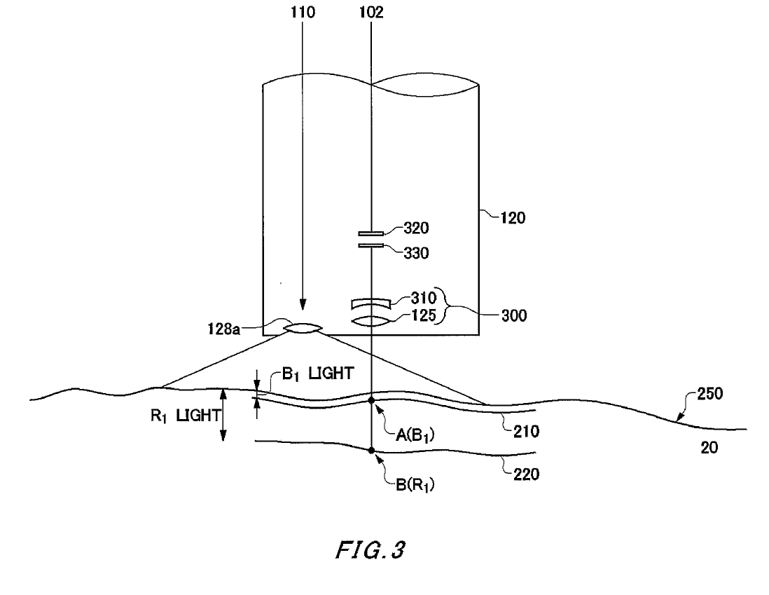

FIG. 3 is a schematic view of exemplary configurations of a light emitting system and an image capturing system in the insertion section 120. The special observation light from the light source 110 passes through the irradiating section 128 to irradiate the analyte 20. The narrow-band blue light (B₁ light) in the special observation light penetrates to the depth d1 from the surface 250 and the narrow-band red light (R₁ light) in the special observation light penetrates to the depth d2, which is greater than the depth d1, from the surface 250.

The image capturing section 124 includes a light receiving section 320 and a wavelength filter section 330. The image capturing optical system 300 has optical characteristics to focus the narrow-band blue light from point A and the narrow-band red light from point B at substantially the same position in the direction of the optical axis of the image capturing optical system 300. The light receiving section 320 is provided at this focal position of the image capturing optical system 300. The wavelength filter section 330 is provided near the light receiving section 320 in the optical path of the returned light between the image capturing optical system 300 and the light receiving section 320. The wavelength filter section 330 has light transmission characteristics to selectively pass at least light in the blue wavelength region and light in the red wavelength region.

The narrow-band blue returned light from point A is focused at the light receiving section 320 through the image capturing optical system 300. The narrow-band red light from point B is also focused at the light receiving section 320 through the image capturing optical system 300. As shown in FIG. 3, the narrow-band light from the irradiating section 128 irradiates a relatively large region of the analyte 20. Furthermore, image capturing is performed in a direction substantially perpendicular to the surface 250. In this case, the narrow-band blue returned light from a plane at a depth d1 from the surface 250 is substantially received on the light receiving surface of the light receiving section 320, and the narrow-band red returned light from a plane at a depth d2 from the surface 250 is also substantially received on the light receiving surface of the light receiving section 320. By providing a plurality of light receiving elements on the light receiving surface of the light receiving section 320, a narrow-band blue light image of the plane at a depth d1 from the surface 250 and a narrow-band red light image of the plane at a depth d2 from the surface 250 can both be obtained by a single image capturing. Received light signals indicating the light received by the light receiving elements on the light receiving surface of the light receiving section 320 are supplied to the image generating section 102 as an image capture signal.

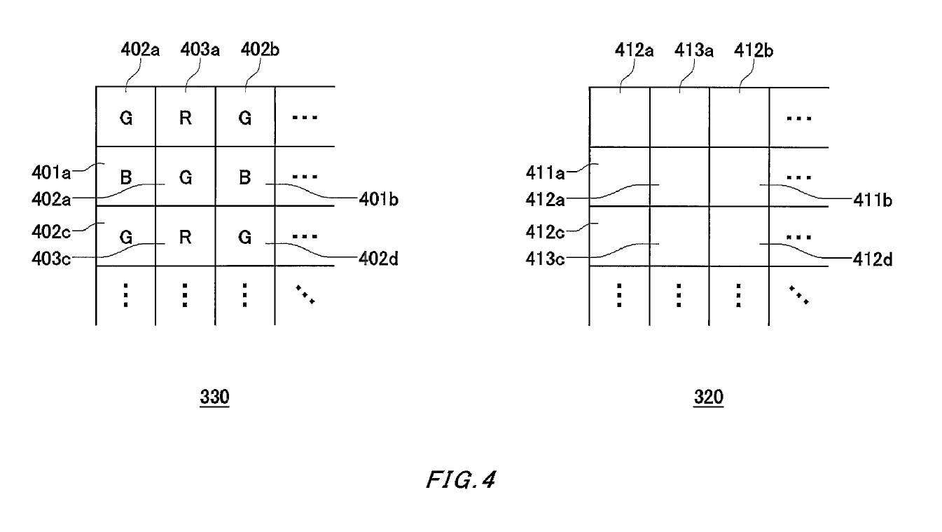

FIG. 4 is a schematic view of exemplary configurations of the wavelength filter section 330 and the light receiving section 320. The wavelength filter section 330 includes a plurality of blue light passing filters 401 that selectively pass light in the blue wavelength region, a plurality of green light passing filters 402 that selectively pass light in the green wavelength region, and a plurality of red light passing filters 403 that pass at least light in the red wavelength region.

In FIG. 4, the blue light passing filters 401a and 401b, green light passing filters 402a to 402d, and red light passing filters 403a and 403c are shown. The blue light passing filter 401a, the two green light passing filters 402a, and the red light passing filter 403a are arranged in a matrix to form one wavelength filter unit. The wavelength filter section 330 may have a wavelength filter array in which a plurality of such wavelength filter units are arranged in a matrix, in the same manner as the light passing filters within a wavelength filter unit. In this way, the wavelength filter section 330 can be formed by arranging blue light passing filters 401, green light passing filters 402, and red light passing filters 403 in a two-dimensional array.

The light receiving section 320 may be formed by arranging a plurality of light receiving elements at positions to selectively receive light passed by the blue light passing filters 401, the green light passing filters 402, and the red light passing filters 403. Specifically, the light receiving section 320 may have a light receiving element array in which a plurality of blue light receiving sections 411 that selectively receive light in the blue wavelength region, a plurality of green light receiving sections 412 that selectively receive light in the green wavelength region, and a plurality of red light receiving sections 413 that receive at least light in the red wavelength region are arranged two-dimensionally.

More specifically, a blue light receiving section 411a receives light passed by a blue light passing filter 401a, a green light receiving section 412a receives light passed by a green light passing filter 402a, and a red light receiving section 413a receives light passed by a red light passing filter 403a. In this way, the blue light receiving sections 411, green light receiving sections 412, and red light receiving sections 413 can respectively be positioned to correspond to blue light passing filters 401, green light passing filters 402, and red light passing filters 403. Each light receiving element may be an image capturing element, such as a CCD or a CMOS.

Here, in addition to light in the red wavelength region, the red light passing filters 403 can also pass the wavelength region of the fluorescent light emitted by the ICG. In other words, the red light passing filters 403 selectively pass light in the red wavelength region and in the fluorescent light wavelength region emitted by the ICG. Therefore, when the analyte 20 is irradiated with excitation light for exciting the ICG, the fluorescent light emitted by the ICG can be received by the red light receiving sections 413 through the red light passing filters 403. Accordingly, the image capturing section 124 can use the red light receiving sections 413 to capture the fluorescent light images with the fluorescent light emitted by the ICG. Furthermore, the fluorescent light emitted by NADH can be received by the blue light receiving sections 411 through the blue light passing filters 401. Accordingly, the image capturing section 124 can use the blue light receiving sections 411 to capture the fluorescent light images with the fluorescent light emitted by the NADH.

In the present embodiment, the blue light passing filters 401 are examples of first wavelength filters that pass light in the wavelength region of the first returned light, and the red light passing filters 403 are examples of second wavelength filters that pass light in the wavelength region of the second returned light. Furthermore, the blue light receiving sections 411 are examples of first light receiving elements that receive light passed by the first wavelength filters, and the red light receiving sections 413 are examples of second light receiving elements that receive light passed by the second wavelength filters.

The image generating section 102 generates images at different positions within the analyte 20 on the optical axis of the image capturing optical system 300, based on the narrow-band blue retuned light and the narrow-band red returned light received by the light receiving section 320. More specifically, the image generating section 102 generates narrow-band blue light images using the narrow-band blue returned light, based on the image capture signals of the blue light receiving sections 411 that received the narrow-band blue returned light. The narrow-band blue light images show regions near the depth d1. Furthermore, the image generating section 102 generates narrow-band red light images using the narrow-band red returned light, based on the image capture signals of the red light receiving sections 413 that received the narrow-band red returned light. The narrow-band red light images show regions near the depth d2.

If the irradiating section 128 emits illumination light spanning substantially the entire wavelength region of visible light, the image capturing section 124 can generate illumination light images of visible light using the blue light receiving sections 411, the green light receiving sections 412, and the red light receiving sections 413. In this way, the light receiving section 320 can receive returned light from the analyte 20 irradiated with illumination light. The image generating section 102 then generates an image of the surface 250 of the analyte 20, based on the returned light from the analyte 20 illuminated with the illumination light.

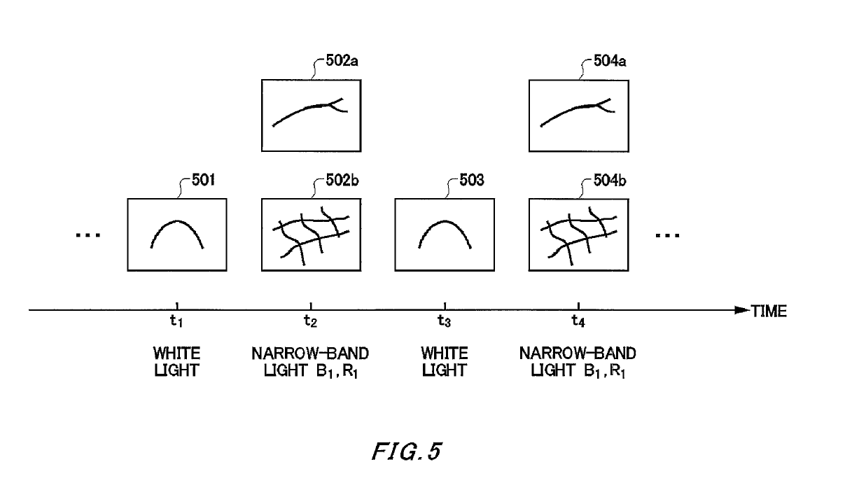

FIG. 5 shows exemplary image capturing timings of the illumination light images and narrow-band light images by the image capturing section 124. The image capturing section 124 is controlled by the control section 104 to switch over time between capturing illumination light images and capturing narrow-band light images. In the example of FIG. 5, the image capturing section 124 captures an illumination light image 501, narrow-band light images 502, an illumination light image 503, narrow-band light images 504, etc. at the times t1, t2, t3, t4, etc.

During the exposure period at the image capturing timing of t1, the control section 104 causes white light to be irradiated as illumination light from the irradiating section 128a toward the analyte 20. When this exposure period is finished, the control section 104 switches the irradiation light from the white illumination light to the narrow-band light, and causes the narrow-band light to be irradiated from the irradiating section 128a toward the analyte 20 during the exposure period at the image capturing timing of t2.

Next, the control section 104 switches the irradiation light from the narrow-band light to white illumination light, and causes the white illumination light to be irradiated from the irradiating section 128a toward the analyte 20 during the exposure period at the image capturing timing of t3. After this, the control section 104 switches the irradiation light from the white illumination light to the narrow-band light, and causes the narrow-band light to be irradiated from the irradiating section 128a toward the analyte 20 during the exposure period at the image capturing timing of t4. As a result of the control section 104 repeating the irradiation light switching operation, the analyte 20 is alternately irradiated by illumination light and narrow-band light over time.

The control section 104 exposes the light receiving section 320 to the image capturing section 124 at each exposure period from t1 to t4, and outputs the acquired image capture signals from the light receiving section 320 to the image generating section 102. The image generating section 102 generates the illumination light image 501 based on the image capture signals from each of the blue light receiving sections 411, green light receiving sections 412, and red light receiving sections 413 acquired at the image capturing timing t1. The image generating section 102 generates the narrow-band light image 502b using narrow-band blue returned light, based on the image capture signals of the blue light receiving sections 411 acquired at the image capturing timing t2, and generates the narrow-band light image 502a using narrow-band red returned light, based on the image capture signals of the red light receiving sections 413 acquired at the image capturing timing t2.

Next, the image generating section 102 generates the illumination light image 503 based on the image capture signals from each of the blue light receiving sections 411, green light receiving sections 412, and red light receiving sections 413 acquired at the image capturing timing t3. The image generating section 102 generates the narrow-band light image 504b using narrow-band blue returned light, based on the image capture signals of the blue light receiving sections 411 acquired at the image capturing timing t4, and generates the narrow-band light image 504a using narrow-band red returned light, based on the image capture signals of the red light receiving sections 413 acquired at the image capturing timing t4.

When switching the irradiation light from the illumination light to the narrow-band light, the control section 104 may continue to drive the visible light source to emit light and insert, into the optical path from the visible light source, a wavelength filter that blocks light that is not in the wavelength region of the narrow-band blue light or the narrow-band red light and that passes light in the wavelength region of the narrow-band blue light and the narrow-band red light. This wavelength filter can be realized by a filter whose light transmission characteristics can be electrically controlled, such as a liquid crystal filter. The control section 104 can alternate between the illumination light and the narrow-band light by electrically controlling the light transmission characteristics of the filter.

The light source 110 may include an LED as the illumination light source and another LED as the narrow-band light source. When switching the irradiation light from the illumination light to the narrow-band light, the control section 104 may stop driving the LED serving as the illumination light source and drive the LED serving as the narrow-band light source. When switching the irradiation light from the narrow-band light to the illumination light, the control section 104 may stop driving the LED serving as the narrow-band light source and drive the LED serving as the illumination light source.

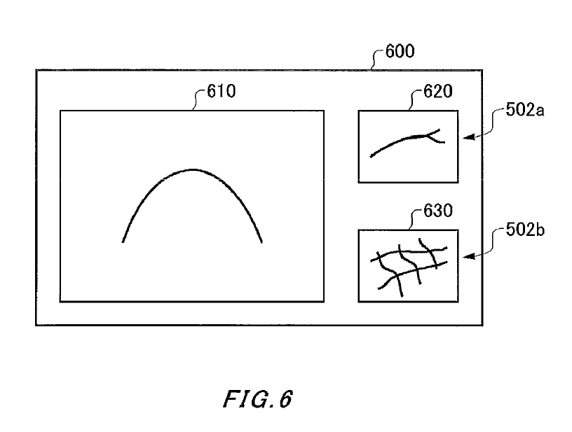

FIG. 6 shows an exemplary image on a screen of the display apparatus 140. The image generating section 102 generates a view in the display area 610 of the screen 600 of the display apparatus 140 that sequentially changes between the illumination light image 501, the illumination light image 503, etc. Furthermore, the image generating section 102 generates a view in the display area 620 of the screen 600 of the display apparatus 140 that sequentially switches between the narrow-band light image 502a, the narrow-band light image 504a, etc. and a view in the display area 630 of the screen 600 of the display apparatus 140 that sequentially switches between the narrow-band light image 502b, the narrow-band light image 504b, etc.

The observer can observe a natural image such as seen by the naked eye from the tip of the insertion section 120, using the visible light view displayed in the display area 610. The observer can be made aware of relatively deep blood vessels in the analyte 20 by the narrow-band light image 502a displayed in the display area 620. The observer can be made aware of capillary blood vessels or the like relatively near the surface layer of the analyte 20 by the narrow-band light image 502b displayed in the display area 630.

The image generating section 102 generates the narrow-band light image 502b and the narrow-band light image 502a in different colors. For example, the image generating section 102 may generate the narrow-band light image 502b such that the intensity of the received narrow-band blue returned light is indicated by the strength of a first color and generate the narrow-band light image 502a such that the intensity of the received narrow-band red returned light is indicated by the strength of a second color. The first color may be a bluish color and the second color may be a reddish color, for example. When viewing the organism with the naked eye, objects on the top layer may appear blue. Therefore, by using a bluish color to represent the narrow-band light image 502b obtained when focusing on objects closer to the surface and using a reddish color to represent the narrow-band light image 502a obtained when focusing on deeper objects, the observer can see a special observation light image that seems natural.

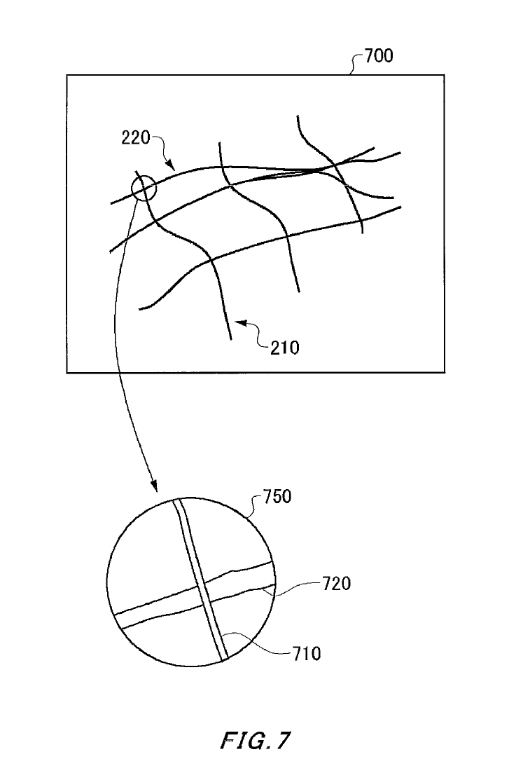

FIG. 7 shows another exemplary narrow-band light image generated by the image generating section 102. The image generating section 102 may generate the composite image 700 based on the narrow-band light image 502a and the narrow-band light image 502b, by superimposing the narrow-band light image 502a and the narrow-band light image 502b on each other. The image generating section 102 may supply the composite image 700 to at least one of the display apparatus 140 and the recording apparatus 150 as an image to be displayed.

The enlarged portion 750 of FIG. 7 shows an object 710 extracted based on the image content of the narrow-band light image 502b and an object 720 extracted based on the image content of the narrow-band light image 502a. The object 710 represents a blood vessel 210 relatively near the surface layer and the object 720 represents a blood vessel 220 in a relatively deep portion. The image generating section 102 may generate the composite image 700 such that the extracted object 710 is emphasized more than the extracted object 720.

Specifically, the image generating section 102 may generate the composite image 700 such that the pixel values representing the object 710 are given more weight than the pixel values representing the object 720. More specifically, with I1(x, y) representing the pixel value of each pixel in the narrow-band light image 502b and I2(x, y) representing the pixel value of each pixel in the narrow-band light image 502a, the image generating section 102 may calculate corresponding pixel values I(x, y) in the composite image 700 such that I(x, y)=α×I1(x, y)+β×I2(x, y), where α>β.

As shown in the enlarged portion 750, for example, the image generating section 102 may overwrite the object 720 with the object 710. As a result, the composite image 700 can be generated to appropriately show the overlapping state of the objects in the analyte 20. When generating the composite image 700, the above process for emphasizing the object 710 more than the object 720 is particularly useful at borders between the object 710 and the object 720. With this process, the vertical positional relationship of the object 710 and the object 720 can be appropriately displayed in the composite image 700, and the observer can be clearly shown that the deep blood vessel represented by the object 720 is deeper than the capillary blood vessel on the surface layer represented by the object 710.

The image generating section 102 may generate the composite image 700 such that the narrow-band light image 502b and the narrow-band light image 502a have different colors. For example, the image generating section 102 may generate the composite image 700 such that narrow-band light image 502b is represented by pixel values of a first color and the narrow-band light image 502a is represented by pixel values of a second color. Here as well, the first color may be a bluish color and the second color may be a reddish color. For a composite image 700 using different colors, the image generating section 102 may emphasize the object 710 more than the object 720. For example, the image generating section 102 may generate the composite image 700 such that the pixel values representing the object 710 are given more weight than the pixel values representing the object 720. The image generating section 102 may generate the composite image 700 such that the object 720 is overwritten by the object 710.

In the above description, the endoscope apparatus 10 operates using mostly narrow-band blue light and narrow-band red light as examples of irradiation light with different penetration depths. As another example, excitation light that excites a fluorescent substance may be used as at least one of the types of irradiation light having different penetration depths. For example, when the analyte 20 contains a fluorescent substance that emits fluorescent light as a result of being excited by light in the second wavelength region, the light in the second wavelength region may be excitation light. In this case, the image capturing optical system 300 focuses the first returned light and the second returned light, which is luminescent light emitted by the fluorescent substance, at substantially the same position on the optical axis.

If the fluorescent substance contained in the analyte 20 emits fluorescent light as a result of excitation by the light in the first wavelength region and the light in the second wavelength region contained in the irradiation light, the light in the first wavelength region and the light in the second wavelength region can also serve as excitation light. In this case, the image capturing optical system 300 focuses both the first returned light and the second returned light, which are in the wavelength region of the fluorescent light emitted by the fluorescent material, at substantially the same position in the direction of the optical axis. Here, the light in the first wavelength region and the light in the second wavelength region contained in the irradiation light may respectively excite different fluorescent substances in the analyte 20.

The function of the control apparatus 100 described above may be realized by a computer. Specifically, by installing a program realizing the function of the control apparatus 100 in a computer, the computer may function as the image generating section 102 and the control section 104. This program may be stored in a computer readable storage medium such as a CD-ROM or a hard disk, and may be provided to the computer by having the computer read this storage medium. Instead, the program may be provided to the computer via a network.

While the embodiments of the present invention have been described, the technical scope of the invention is not limited to the above described embodiments. It is apparent to persons skilled in the art that various alterations and improvements can be added to the above-described embodiments. It is also apparent from the scope of the claims that the embodiments added with such alterations or improvements can be included in the technical scope of the invention.

The operations, procedures, steps, and stages of each process performed by an apparatus, system, program, and method shown in the claims, embodiments, or diagrams can be performed in any order as long as the order is not indicated by “prior to,” “before,” or the like and as long as the output from a previous process is not used in a later process. Even if the process flow is described using phrases such as “first” or “next” in the claims, embodiments, or diagrams, it does not necessarily mean that the process must be performed in this order.

What is claimed is:

1. An endoscope apparatus that captures an image of a target using returned light from the target irradiated with irradiation light, the endoscope apparatus comprising:
an irradiating section that irradiates the target with the irradiation light containing light in a first wavelength region, which reaches a first penetration depth within the target, and light in a second wavelength region, which reaches a second penetration depth within the target;
an optical system that focuses, at substantially the same position in a direction of an optical axis thereof; first returned light from a position at a distance of the first penetration depth from a surface of the target in a direction of emission of the light in the first wavelength region and second returned light from a position at a distance of the second penetration depth from the surface of the target in a direction of emission of the light in the second wavelength region; and
a light receiving section that receives the first returned light and the second returned light focused by the optical system.

2. The endoscope apparatus according to claim 1, further comprising an image generating section that generates images within the target at different positions in the direction of the optical axis of the optical system, based on the first returned light and the second returned light received by the light receiving section.

3. The endoscope apparatus according to claim 2, wherein
the irradiating section includes a light source that emits (i) illumination light for illuminating the surface of the target, (ii) the light in the first wavelength region, which is light in a narrower wavelength region than the illumination light, and (iii) the light in the second wavelength region, which is light in a narrower wavelength region than the illumination light,
the light receiving section further receives returned light from the target irradiated with the illumination light, and
the image generating section further generates an image of the surface of the target based on the returned light from the target irradiated with the illumination light.

4. The endoscope apparatus according to claim 3, wherein
the illumination light is light in a visible region,
the light in the first wavelength region is light in a blue wavelength region, and
the first returned light is light in substantially the same wavelength region as the first wavelength region.

5. The endoscope apparatus according to claim 4, wherein
the light in the second wavelength region is light in a longer wavelength region than the first wavelength region, and
the second returned light is light in substantially the same wavelength region as the second wavelength region.

6. The endoscope apparatus according to claim 4, wherein
the light in the second wavelength region is light in an infrared region.

7. The endoscope apparatus according to claim 1, further comprising a first wavelength filter that passes light in a wavelength region of the first returned light and a second wavelength filter that passes light in a wavelength region of the second returned light, wherein
the light receiving section includes a first light receiving element that receives light passed by the first wavelength filter and a second light receiving element that receives light passed by the second wavelength filter.

8. The endoscope apparatus according to claim 7, wherein
a plurality of the first wavelength filters and a plurality of the second wavelength filters are provided, and are arranged two-dimensionally, and
a plurality of the first light receiving elements and a plurality of the second light receiving elements are provided at positions corresponding respectively to the first wavelength filters and the second wavelength filters.

9. The endoscope apparatus according to claim 1, wherein
the target includes a luminescent substance that emits luminescent light as a result of being excited by the light in the second wavelength region contained in the irradiation light, and
the optical system focuses the first returned light and the second returned light, which is the luminescent light emitted by the luminescent substance, at substantially the same position in the direction of the optical axis.

10. The endoscope apparatus according to claim 9, wherein
the luminescent substance emits luminescent light as a result of being excited by the light in the first wavelength region in the irradiation light and also emits luminescent light as a result of being excited by the light in the second wavelength region in the irradiation light, and
the optical system focuses the first returned light and the second returned light, which are both in the wavelength region of the luminescent light emitted by the luminescent substance, at substantially the same position in the direction of the optical axis.

11. The endoscope apparatus according to claim 9, wherein
the light in the first wavelength region and the light in the second wavelength region included in the irradiation light respectively excite different luminescent substances in the target.

12. The endoscope apparatus according to claim 9, further comprising an injecting section that injects each of a plurality of the luminescent substances into the target.
